(19) United States
(12) Patent Application Publication
(10) Pub. No.: US 2004/0076957 A1
(43) Pub. Date: Apr. 22, 2004

(54) Hybridized array analysis aiding method and analysis aiding service

(76) Inventor: Tetsuhiko Yoshida, Aichi (JP)

Correspondence Address: Oliff & Berridge, Suite 500, 277 South Washington Street, Alexandria, VA, 22314, US

(21) Appl. No.: 10/250,921

(22) PCT Filed: Feb. 1, 2002
(86) PCT No.: PCT/JP02/00832

(30) Foreign Application Priority Data
Feb. 1, 2001 (JP) 2001-25096

(51) Int. Cl. C12Q 1/68; G06F 19/00; G01N 33/48; G01N 33/50

(52) U.S. Cl. 435/6; 702/20

(57) ABSTRACT

To provide a technology that facilitates extraction of significant information from a color matrix that is present on a hybridized array 1.

In order to facilitate analysis of the array after the hybridization of targets with the array in which probes were two-dimensionally disposed in the form of spots on a substrate, color information of the spots on the array is stored. Subsequently, based upon the color information, display 48 is output. Display 48 shows a parallel arrangement of color matrices, which parallel arrangement of color matrices comprises at least color matrix 36 formed from n columns (n is a natural number), color matrix 38 formed from n+k columns (k is a natural number), and color matrix 40 formed from n+2k columns.

Thus, a latent characteristic in the color matrix on the array, which previously could not be recognized, distinctly appeared in display 48 of the parallel arrangement of matrices. Accordingly, the recognition of the characteristic became easy.

### BACKGROUND OF THE INVENTION

1. Field of the Invention

The present invention relates to techniques for facilitating analysis of arrays after hybridization of substances (generally speaking, targets such as substances originating from living things) with probes on the array. The array is made by two-dimensionally disposing the probes in the form of spots on a substrate. Examples of such arrays are DNA microarrays, DNA chips, protein arrays and tissue arrays.

2. Description of the Related Art

The nucleotide base sequences of the human genome has been completely decoded. Analysis of the significance and functions of the genetic information has started and is still continuing.

Therefore, analyzing technologies, e.g., DNA microarrays, have been developed.

In these array technologies, various probes (e.g., DNA, oligo DNA, protein, or tissue) are two-dimensionally disposed and affixed on a substrate in the form of spots. A target-containing solution is utilized together with the array. The target-containing solution contacts the array. Typically, the targets hybridize with some probes and do not hybridize with other probes. By analyzing the results of which probes hybridize relative to the targets, the functions of the targets and probes can be further studied. In this specification, an array having bound targets will be referred to as a “hybridized array.” Some probes on the array have hybridized with the targets and other probes do not hybridize with the targets.

In order to determine whether or not the targets have hybridized with the probes, the targets are first colorized. Generally, the targets are bound to a fluorescent dye (i.e. the targets are labeled with the fluorescent dye) in advance. By measuring the intensity of the color of each spot on the hybridized array, it can be determined whether or not the colored target has hybridized with the probe.

In this specification, the term “color” of each spot has a broader meaning than the common meaning of the term “color.” The term “color” as used in the specification includes a color that can be visually perceived when the spot is observed with the assistance of a microscope or other optical instrument, if the targets have been colorized with a coloring substance that can be identified using the microscope or other optical instrument. In this case, the color that is visually perceived using the microscope or the like is a color that is commonly recognized. If the targets have been colorized with a coloring substance that emits light when reference light is shined onto the substance, the color of the spot, which color can be visually perceived when the substance is exposed to the reference light, is also called a color. If the targets are colored with a coloring substance that emits light when exposed to excitation light having a specific wavelength, the color of the excitation light that causes the coloring substance to emit light is also called a spot color for convenience. If the wavelength of the excitation light exists in the non-visible region, a color is obtained by converting the wavelength in the non-visible region into a wavelength existing in the visible region according to a predetermined rule and also will be called a color according to this specification. For instance, the above-described color definition will be utilized when a spot colored with a fluorescent dye that emits white light when exposed to ultraviolet light having a short wavelength is called “blue” for the sake of convenience. Similarly, the above described color-definition is also utilized when a spot colored with a fluorescent dye that emits white light when exposed to ultraviolet light having a long wavelength is called “red” for the sake of convenience.

In this specification, information concerning a wavelength, which information can be utilized in order to determine optically whether or not a colored target has hybridized with a spot probe, will be referred to as a color. If a spot that emits white light when ultraviolet light having a short wavelength is directed to the spot is regarded as a “blue spot” for convenience, the blue spot indicates that the target has hybridized with the probe. If the spot does not emit white light and, therefore, is regarded as a “non-blue spot” for convenience, the non-blue spot indicates that the target has not hybridized with the probe. In this case, the above-described color definition also will be utilized.

Typically, two types of labeled targets are utilized to react with an array. For instance, target A is colored with a fluorescent dye that emits light when exposed to excitation light A, and target B is colored with a fluorescent dye that emits light when exposed to excitation light B; targets A and B are simultaneously brought into contact with the array.

In this case, if normal genes are colored green (i.e., the normal genes are bound to a fluorescent dye that emits light when exposed to green excitation light) and if non-normal genes are colored red, the spots will become green where probes have hybridized with only normal genes (i.e., the spots will appear bright when green excitation light is directed to the dye); spots will become red where the probes have hybridized with non-normal genes; spots will become yellow where the probes have hybridized with both types of genes.

According to this research, the probes affixed in the red spots can be utilized as markers for the non-normal genes. In the alternative, by preparing an array on which the probes affixed in the red spots are arranged, it can be determined whether or a gene extracted from a specimen is non-normal. Such methods indicate diagnostic improvement.

Array probe density has been increasing. Nowadays, more than ten thousand types of probes can be disposed on a single ordinary glass slide. Higher probe density can be obtained using DNA chips that are fabricated by synthesizing probes on a substrate using photo-lithography techniques. Such chips enable simultaneous testing of a large number of probes. In practice, numerous types of probes must be tested in order to study the functions of targets originating from living things.

Thus far, analytic techniques have not yet advanced for extracting significant information from test results of numerous types of probes. However, if the test results are successfully analyzed, knowledge will be obtained that can be utilized very effectively.

If the number of columns of spots is A and the number of rows of spots is B in an array having two-dimensionally arranged spots, a color matrix (A columns·B rows) will be obtained by detecting the colors of the spots on the hybridized array. Nowadays, researchers are trying to analyze the matrix probes in order to extract significant information. However, extracting such information is very difficult and has been a large obstacle in research.

In order to overcome this obstacle, attempts have been made to convert a color matrix (A columns·B rows) into a more effective form. For instance, researchers at Stanford University and NIH in the United States have proposed converting the color matrix (A columns·B rows) into a one-dimensional color matrix; then, the one-dimensional color matrix is further converted into another matrix in which test results corresponding to the same types of targets are repeatedly and laterally positioned. In this case, if the number of same type of targets is C, a color matrix is obtained by this conversion that provides C columns and A×B rows or (C columns·A×B rows).

By converting the color matrix (A columns·B rows) to another color matrix (C columns·A×B rows) in such manner, color patterns that are common to the same type of targets will distinctly appear, thereby enabling researchers to easily recognize the common characteristics of the same type of targets.

### DISCLOSURE OF THE INVENTION

However, only a limited number of characteristics can be easily recognized by converting the color matrix (A columns·B rows) into another color matrix (C columns·A×B rows). Substantial unknown information still remains unanalyzed.

It is, accordingly, one object of the present invention to overcome problems in the known art in order to effectively facilitate extraction of significant information from a color matrix that is present on a hybridized array.

The invention positively utilizes the excellent ability of humans to recognize patterns or to extract characteristics. Thus far, it is still believed that the ability of humans is superior to the ability of computers in this regard. In the present invention, conversion is performed in order to provide a display, and much unknown information embedded within the array (e.g., regularity repeating genetic information) can be extracted from the display by using the recognition abilities of humans.

Specifically, the present invention provides methods for converting a color matrix that is present on the hybridized array into a display that facilitates pattern recognition or characteristic extraction.

In these methods, information concerning the colors of the spots on the hybridized array is displayed such that a color matrix formed from n columns, a color matrix formed from (n+k) columns, and a color matrix formed from (n+2k) columns are arranged in parallel with each other. Herein, n and k are natural numbers. The number of color matrices that are arranged in parallel may be three or more, in the case of which a color matrix formed from (n+3k) columns, a color matrix formed from (n+4k) columns, and so on are arranged in parallel with each other.

The natural number n may be 1, but is not limited to 1. Similarly, the natural number k may be 1, but is not limited to 1.

The method for displaying the information is not particularly limited. Matrices may be erasably displayed using a CRT, a liquid crystal display, a plasma display, or various types of projectors. The display of the matrices may be printed on paper, film, printing paper, etc by using a printer or an exposure device.

In the finally displayed parallel arrangement of color matrices, the average color of each individual spot on the array may be displayed. In the alternative, the color distribution of each spot may be displayed as is.

The colors of the spots on the array may be displayed in the form of a particular shape (e.g., square, rectangle, circle, or rhombus). In the alternative, the color(s) may be displayed in the form of an actual shape of the corresponding, light-emitting spot.

The colors of the finally displayed parallel arrangement of color matrices may be different from the colors of the corresponding spots on the array (i.e., as stated above, the term “color” used herein may have a slightly different meaning from the term “color” as commonly used). For example, color information of each spot on the array, which color information is sometimes the same as background information of an array substrate or which color information sometimes includes background information of an array substrate, may be converted into a color using a variety of computations. Excitation light may be non-visible light. Color(s) may be displayed that was obtained by converting the wavelength of the excitation light into the wavelength of the visible light according to a particular rule.

In the present invention, the terms “columns and rows” of a matrix have broader meaning than the ordinary mathematical definitions. Although a “column” usually extends in the vertical direction and a “row” extends in the horizontal direction, this relationship may be reversed.

As was disclosed in Japanese Laid-open Patent Publication No. 11-066040, hidden, unrecognized characteristics noticeably appear by positioning, in parallel, a color matrix formed from n columns, a color matrix formed from (n+k) columns, and a color matrix formed from (n+2k) columns.

According to the invention described in the above publication, latent characteristics in the color matrix (A columns·B rows) on the array distinctly appear in the parallel display of the color matrix formed from n columns, the color matrix formed from (n+k) columns, and the color matrix formed from (n+2k) columns, (a color matrix formed from (n+3k) columns, a color matrix formed from (n+4k) columns, may come thereafter). This effectively facilitates characteristic extraction.

The present invention can be embodied in an apparatus for facilitating analysis of hybridized arrays. This facilitating apparatus includes a means for storing color information for each spot disposed on a hybridized array. The apparatus also includes a means for generating from the stored color information a parallel display of at least a color matrix formed from n columns, a color matrix formed from (n+k) columns, and a color matrix formed from (n+2k) columns.

Typically, the ratio between light intensities is utilized as color information, which respective light intensities are detected as emitted excitation light having two different wavelengths. For instance, when an array is hybridized with green-labeled targets and red-labeled targets, (intensity of emitted red wavelength light)/(intensity of emitted green wavelength light+intensity of emitted red wavelength light), or (intensity of emitted green wavelength light)/(intensity of emitted green wavelength light+intensity of emitted red wavelength light) is stored as color information.

In the present invention, if the targets are labeled using a single color, each color intensity is also considered to be a color. For instance, if a spot having a dark color, a spot having a light color, and a spot having an unrecognized color are present, the intensities of the color are considered to be colors.

The average of the color information of each spot may be stored. In the alternative, the spot may be divided into very small areas (i.e., pixels) and the color information of each very small area may be stored.

The display means may be a temporary display means (e.g., CRT, liquid crystal display, plasma display, or various projectors). In the alternative, a printer or exposure device, which prints on paper, film, printing paper, etc can also be utilized. Such display means may display the spots on the array by using a regular shape or by using the actual shape of the corresponding, light-emitting spot.

This apparatus enables hidden, non-obvious characteristics the color matrix on the array (A columns·B rows) to appear prominently in the output display.

Accordingly, the process for extracting characteristics becomes very easy.

### BRIEF DESCRIPTION OF THE DRAWINGS

FIG. 1 schematically shows a process for analyzing a hybridized array.

FIG. 2 shows a detailed process for measuring the colors of spots.

FIG. 3 shows an example of a color matrix displaying measured colors of the spots.

FIG. 4 shows a first example in which measured colors of the spots are reproduced in an array.

FIG. 5 shows a second example in which measured colors of the spots are reproduced in an array.

FIG. 6 shows a third example in which measured colors of the spots are reproduced in an array.

FIG. 7 shows a fourth example in which measured colors of the spots are reproduced in an array.

FIG. 8 shows a fifth example in which measured colors of the spots are reproduced in an array.

FIG. 9 shows a sixth example in which measured colors of the spots are reproduced in an array.

FIG. 10 shows a first example of a color matrix in which a converted arrangement of the measured colors of the spots is repeatedly positioned.

FIG. 11 shows a second example of a color matrix in which a converted arrangement of the measured colors of the spots is repeatedly positioned.

FIG. 12 shows a third example of a color matrix in which a converted arrangement of the measured colors of the spots is repeatedly positioned.

FIG. 13 shows a fourth example of a color matrix in which a converted arrangement of the measured colors of the spots is repeatedly positioned.

FIG. 14 shows a fifth example of a color matrix in which a converted arrangement of the measured colors of the spots is repeatedly positioned.

FIG. 15 shows a sixth example of a color matrix in which a converted arrangement of the measured colors of the spots is repeatedly positioned.

FIG. 16 is a first example in which the array of the measured colors of the spots is converted into a parallel arrangement of color matrices that have different numbers of columns.

FIG. 17 is a second example in which the array of the measured colors of the spots is converted into a parallel arrangement of color matrices that have different numbers of columns.

FIG. 18 is a third example in which the array of the measured colors of the spots is converted into an arrangement of color matrices that have different numbers of columns.

FIG. 19 is a fourth example in which the array of the measured colors of the spots is converted into a parallel arrangement of color matrices that have different numbers of columns.

FIG. 20 is a fifth example in which the array of the measured colors of the spots is converted into a parallel arrangement of color matrices that have different numbers of columns.

FIG. 21 is a sixth example in which the array of the measured colors of the spots is converted into an arrangement of color matrices that have different numbers of columns.

FIG. 22 is a seventh example in which measured colors of the spots are reproduced in an array.

FIG. 23 is a seventh example in which the array of the measured colors of the spots is converted into an arrangement of color matrices that have different numbers of columns.

### BEST MODE FOR PRACTICING THE INVENTION

For example, if 100 arrays are hybridized with 100 types of targets extracted from a normal cell and result in identical matrices and if another 100 arrays are hybridized with 100 types of targets extracted from an non-normal cell and also result in the identical matrices and if the group of color matrices corresponding to the normal targets are different from the group of color matrices corresponding to the non-normal targets, it is easy to determine whether the cell is normal or non-normal by using the color matrices of the hybridized arrays.

However, in practice, 100 types of color matrices are obtained from the 100 arrays that were hybridized with the normal targets and another 100 types of color matrices are obtained from the 100 arrays that were hybridized with the non-normal targets. In this case, even if one type of color matrix is carefully observed, whether the cell is normal or non-normal cannot be determined. All 200 types of color matrices should be compared with one another in order to extract common differences that are found between the 100 types of matrices corresponding to the normal targets and the 100 types of matrices corresponding to the non-normal targets. Extracting characteristics that are different between the matrices corresponding to the normal targets is not efficacious. It is necessary to extract characteristics that are different between the group of matrices corresponding to the normal targets and the group of matrices corresponding to the non-normal targets. The extraction of such characteristics permits differentiation between the normal cell and the non-normal cell.

Even if the 200 color matrices, each having A columns and B rows, are compared with each other, it would be extremely difficult to extract characteristics that are different between the group of matrices corresponding to the normal targets and the group of matrices corresponding to the non-normal targets, but are not different between the matrices corresponding to the normal targets.

According to one embodiment of the present invention, a display of 200 types of matrices is obtained. In the display, a color matrix formed from n columns, a color matrix formed from (n+k) columns, and a color matrix formed from (n+2k) columns are positioned in parallel to each other.

The resulting display distinctly shows characteristics that are common to matrices corresponding to the normal targets, characteristics that are common to the matrices corresponding to the non-normal targets, characteristics that are found in the matrices corresponding to the non-normal targets but not found in the matrices corresponding to the normal targets, and characteristics that are found in the matrices corresponding to the normal targets but not found in the matrices corresponding to the non-normal targets. Accordingly, recognition of the characteristics becomes easy.

If the spots on the color matrix on the array (A columns·B rows) are respectively represented by S1-1, S1-2, • • • , S2-1 • • • (herein, a numeral at the beginning of each code represents a position number in the row direction and a numeral at the end of each code represents a position number in the column direction), one column (one column·A×B rows) can be obtained by sequentially aligning the spots S1-1 [symbol] S1-2 • • • S1-A [symbol] S2-1 [symbol] S2-2 • • • S2-A [symbol] S3-1 [symbol] S3-2 •SB-2 • • • SB-A or by sequentially aligning the spots S1-1 [symbol] S2-1 • • • SB-1 [symbol] S1-2 [symbol] S2-2 • • • SB-2 [symbol] S1-3 [symbol] S2-3 • • • SB-3 [symbol] • • • S1-A [symbol] S be sampled every other one, every other row, or every other column. That is, the spots may be arranged in any order under the constraint that each spot is used once.

After one column of spots is obtained, matrices that are different from each other in the number of columns are arranged in parallel according to various arranging patterns, which are described in Japanese Laid-open Patent Publication No. 11-066040.

The present invention may be practiced in the following forms.

(Form 1) Colors are selected from the spectrum ranging from red to blue in accordance with (red intensity)/(red intensity+green intensity) ratio of each spot that hybridized with two types of targets, which were respectively red-colored and green-colored. The selected colors are respectively shown in the form of quadrangles. The quadrangles in the selected colors are displayed in the form of a matrix.

(Form 2) A matrix of color dots is generated by a computer.

(Form 3) The matrix of color dots, which was generated by the computer, is printed by a printer.

(Form 4) Each of the dots forming the color matrix is rectangular.

(Form 5) All the dots that are arranged in rows and columns have the same shape and size.

(Form 6) Each dot of the color matrix corresponds to a spot.

(Form 7) Each dot of the color matrix corresponds to a pixel.

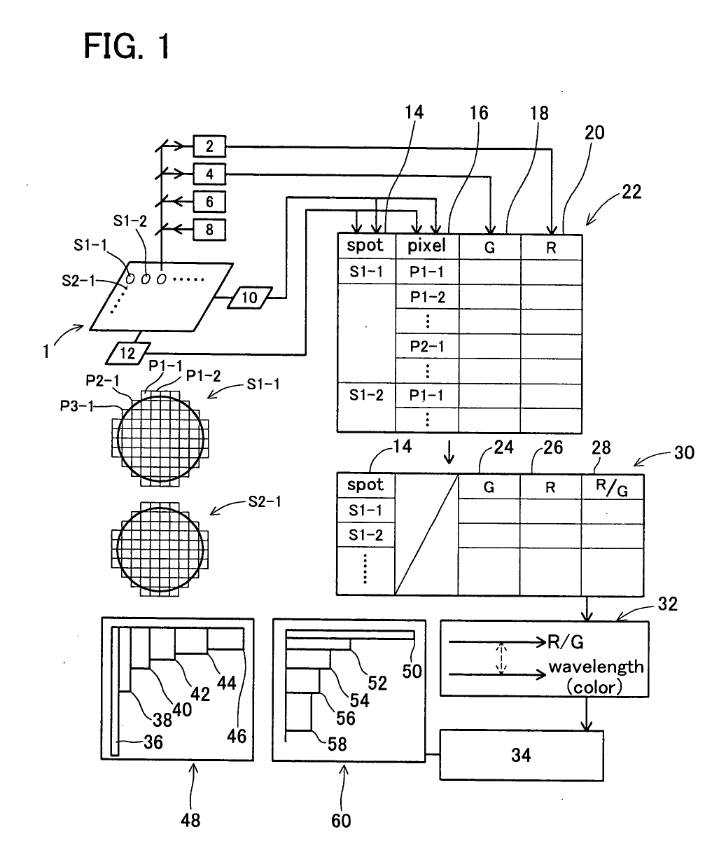

FIG. 1 schematically shows an overall process for analyzing hybridized array 1. Spots S1-1, S1-2, • • • , spots S2-1, • • • , for example, are two-dimensionally disposed on hybridized array 1. A probe has been fixed at each of the spots. Herein, a numeral at the beginning of the code of each spot represents a position number in a vertical direction and a numeral at the end of the code represents a position number in a horizontal direction. Some of the probes hybridize with the targets bound to a fluorescent dye that emits light when exposed to excitation light 6, while others do not hybridize with the targets. Likewise, some of the probes hybridize with the targets bound to a fluorescent dye that emits light when exposed to excitation light 8, while others do not hybridize with the targets.

Hybridized array 1 is placed on a table, which is moved in an X direction by X actuator 10 and is moved in a Y direction by Y actuator 12.

An optical device is installed on the table. The optical device is capable of selecting either excitation light 6 or excitation light 8 and is capable of illuminating the selected light on a very small area of array 1. Light-sensitive detector 2 detects luminescence intensity on the array when excitation light 6 is illuminated on the array. Light-sensitive detector 4 detects luminescence intensity on the array when excitation light 8 is illuminated on the array.

Each image element (pixels) on array 1, which is measured by the optical systems, is much smaller than a spot size. Therefore, the pixel distribution within each spot S can be measured. Pixels P1-1, P1-2, • • • , P2-1, • • • are two-dimensionally disposed. Similarly, the numeral at the beginning of the code of each pixel represents a position number in the vertical direction and a numeral at the end of the code represents a position number in the horizontal direction.

For the sake of convenience, light intensity that is detected by light-sensitive detector 2 when each very small area is illuminated with excitation light 6 is called “red intensity.” Likewise, light intensity that is detected by light-sensitive detector 4 when each very small area is illuminated with excitation light 8 is called “green intensity.”

For each pixel, the red intensity detected by light-sensitive detector 2 and the green intensity detected by light-sensitive detector 4 are stored in memory 22 of a computer. Column 14 of the charts shown in FIG. 1 provides a list of spot positions, each of which is expressed by a position number in the vertical direction and a position number in the horizontal direction. Column 16 of the chart of FIG. 1 provides a list of pixel positions, each of which is expressed by a position number in the vertical direction and a position number in the horizontal direction. These positions are obtained from positional information, which is provided by X actuator 10 and Y actuator 12. Columns 18, 20 of the chart respectively provide a list of green intensities of the pixels and a list of red intensities of the pixels.

The computer includes a program for calculating the average luminescence intensity of each spot by calculating the average of the pixel intensities within the spot, which intensities are stored in memory 22. The calculation result is stored in memory 30. Column 24 of the chart that is shown in FIG. 1 provides a list of the average green intensities of the spots, and column 26 provides a list of the average red intensities of the spots.

The computer also includes a program for calculating the ratio between the red intensity and green intensity of each spot, both of which intensities are stored in memory 30. The calculated ratio (i.e., red intensity/green intensity) is stored in the location that is indicated by reference numeral 28 in FIG. 1.

The computer includes a program for selecting a specific color in accordance with the ratio (red intensity/green intensity) stored in the location indicated by reference numeral 28. According to this program, the computer selects a color. In the present embodiment, the lower the red intensity/green intensity is (i.e., the greater the green intensity is), the bluer the selected color is; the higher the red intensity/green intensity is (i.e., the greater the red intensity is), the redder the selected color is.

Printer 34 is connected to the computer. In correspondence with the spots, the printer 34 prints colored dots that were respectively selected based upon the red intensity/green intensity ratios.

On the array, spots are arranged in A columns (A is the number of columns) and B rows (B is the number of rows). In this case, typically, a matrix of color dots arranged in A columns and B rows, which is a conventional manner of displaying color dots, will be printed by printer 34.

However, in the present embodiment, chart 48 is made. In chart 48, the color dots, the number of which is A×B, are arranged such that color matrix 36 {one column·(A×B)rows}, color matrix 38 {two columns·(A×B)/2 rows}, color matrix 40 {three columns·(A×B)/3 rows}, color matrix 42 {four columns·(A×B)/4 rows}, color matrix 44 {five columns·(A×B)/5 rows}, • • • are arranged in parallel to each other.

If necessary, the horizontal and vertical relationships may be reversed. Such an example is shown in chart 60. If the matrices are arranged vertically instead of horizontally, the above-described explanation applies to this chart as well. Specifically, chart 60 shows a display in which color matrix 50 {one row·(A×B)columns}, color matrix 52 {two rows·(A×B)/2 columns}, color matrix 54 {three rows·(A×B)/3 columns}, color matrix 56 {four rows·(A×B)/4 columns}, color matrix 58 {five rows·(A×B)/5 columns}, • • • are arranged in parallel to each other according to a mathematical definition. That is, both charts contain the same contents.

In chart 48, which is finally displayed, a color matrix formed from the single column is not necessarily required to come at the beginning of the arrangement of matrices. A color matrix formed from n columns (n is a natural number) may come at the beginning of the arrangement. The increments in the number of columns is not necessarily required to be one, but may be k (k is a natural number). The number of matrices that are positioned in parallel with each other is not particularly limited, as long as the number is three or more. Generally, the more color matrices that are positioned, the more obvious the characteristics will become. Generally, 100 or more matrices are arranged in parallel to each other.

The manner for displaying final chart 48 is not particularly limited. By using a CRT, liquid crystal display, plasma display, or various types of projectors, final chart 48 may be temporarily and erasably displayed. In the alternative, final chart 48 may be printed out, e.g., on film, printing paper, or another material in addition to paper by a printer or exposure device.

In this case, the dot color is determined from the red intensity/green intensity ratio of each spot, which ratio has been stored as reference numeral 28 in the computer. Therefore in the parallel arrangement 48 of the color matrices, which is finally displayed, the color corresponding to the average color of each of the spots on the array is displayed. In the alternative, the color dot may be displayed for each pixel, in which case the color distribution within the corresponding spot is also displayed in the form of a color matrix.

The dot color may be determined by the red intensity stored in the location indicated by reference numeral 18 or by the red intensity stored in the location indicated by reference numeral 24. In this case, the dot color is selected from a color ranging from dark red, through light red, to white (or black). White or black is selected when the red intensity is zero. Similarly, the color dot may be determined by the green intensity stored in the location indicated by reference numeral 20 or by the green intensity stored in the location indicated by reference numeral 26. In this case, the color dot is selected from a color ranging from dark green, through light green, to white (or black).

When the dots are displayed in correspondence with the spots on the array, any shape, such as a square, rectangle, circle, or rhombus, may be used as a dot shape. Similarly, when the dots are displayed in correspondence with the pixels on the array, any dot shape may be used. Consequently, each color is displayed in an actual shape of the corresponding spot that is emitting light.

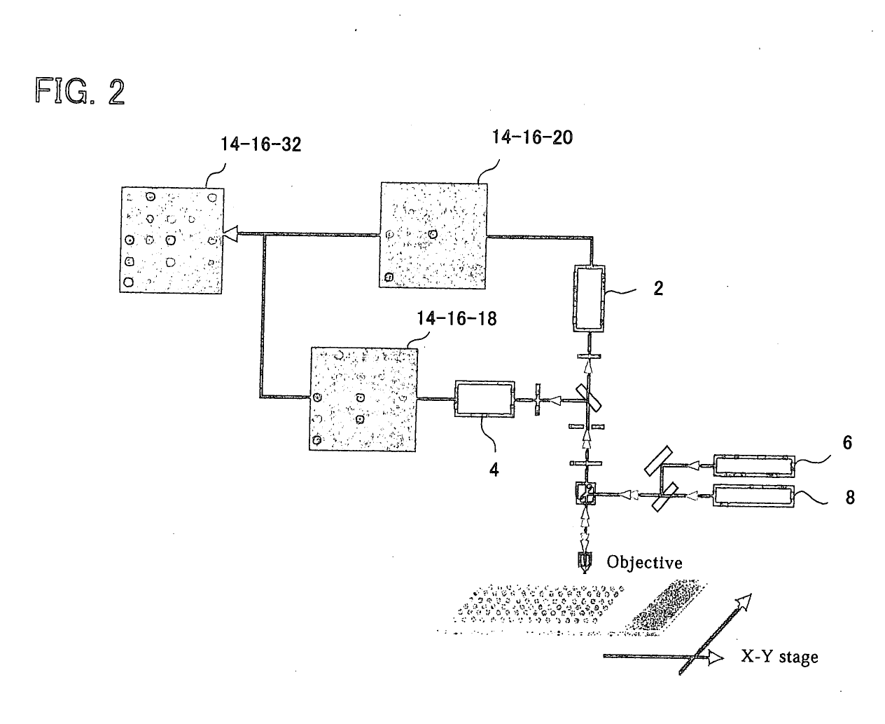

FIG. 2 shows examples of the measurement results of the hybridized array. Reference numeral 14-16-18 represents an example in which a green dot having a color intensity that was determined by the corresponding green intensity is displayed for each pixel. Vivid green dots are displayed for the pixels having high green intensities, dark green dots are displayed for the pixels having low green intensities, and black dots are displayed for the pixels whose green intensities are zero. Reference numeral 14-16-20 represents an example in which a red dot having a color intensity that was determined by the corresponding red intensity is displayed for each pixel. Vivid red dots are displayed for the pixels having high red intensities, dark red dots are displayed for the pixels having low red intensities, and black dots are displayed for the pixels whose red intensities are zero. Reference number 14-16-32 represents an example in which a dot having a color that was determined by the corresponding ratio of the red intensity to the green intensity is displayed for each pixel.

In accordance with the prior art, the positional relationships of the dots or dots corresponding to the pixels exactly correspond to the positional relationships of the spots on the array.

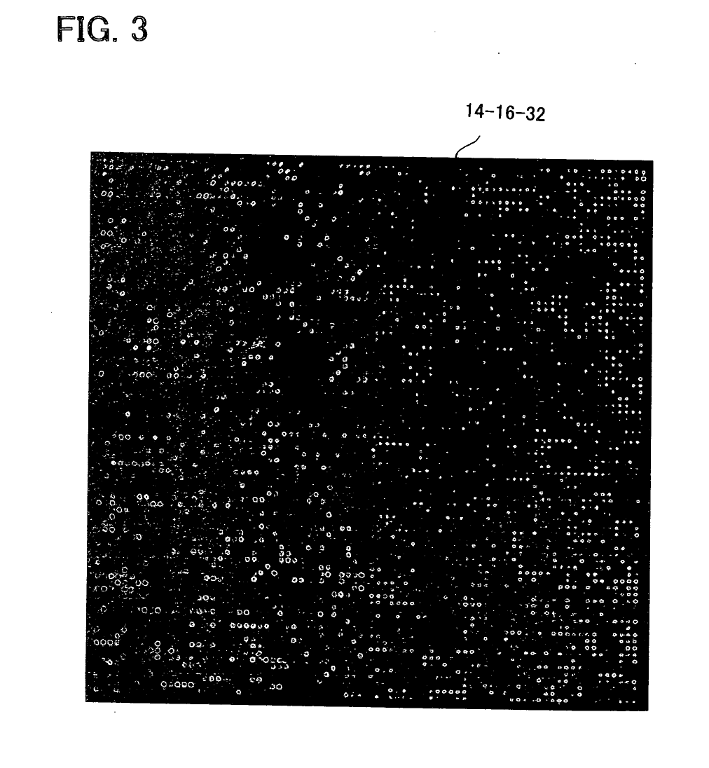

FIG. 3 shows another example in which a dot having a color that was determined by the corresponding ratio of the red intensity to the green intensity is displayed for each pixel. In accordance with the prior art, the positional relationships of the dots corresponding to the pixels exactly correspond to the positional relationships of the spots on the array.

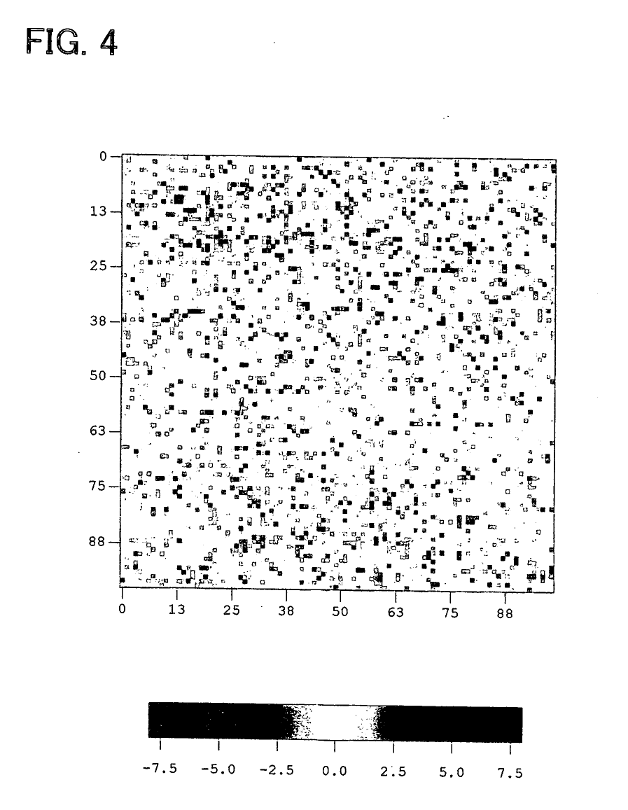

FIG. 4 shows another example in which a dot having a color that was determined by the average ratio of the red intensity to the green intensity is displayed for each spot. The positional relationships of the dots corresponding to the spots exactly correspond to the positional relationships of the spots on the array. The dots each have a quadrangular profile and are arranged in contact with each other.

FIGS. 4 to 9 show examples that were obtained when identical DNA microarrays were used. The DNA microarrays were each hybridized with two types of targets. One of the targets is RNA that was extracted from a normal cell. The RNA was labeled with a fluorescent dye that emits light when exposed to green excitation light. The other is RNA that was extracted from a melanoma cell, which is a type of skin cancer. This RNA is marked with a fluorescent dye that emits light when exposed to red excitation light.

The lower the average ratio of the red intensity to the green intensity is (i.e., the greater the green intensity is) in each spot, the bluer the displayed dot is; the higher the average ratio of the red intensity to the green intensity is (i.e., the greater the red intensity is) in each spot, the redder the displayed dot is. Specifically, the red intensity/green intensity is converted into a logarithm having a base of 2, and the color dot is determined by the value obtained by the conversion. The relationship between the value of the log₂ (red intensity/green intensity) and the dot color is shown at the bottom of each of FIGS. 4 to 9.

In the color matrix of FIG. 4, each of the red dots represents a spot in which a probe that hybridized with the non-normal RNA and did not hybridize with the normal RNA is fixed. Each of the blue dots represents a spot in which a probe that hybridized with the normal RNA and did not hybridize with the non-normal RNA is fixed. Each of the white dots represents a spot in which a probe that hybridized with both the normal RNA and the non-normal RNA is fixed.

Although some of the probes hybridized with the targets and appeared red and others did not hybridize with the targets and did not appear red, regularities in determining whether each gene is non-normal or normal cannot be found from the color matrix. Finding such regularities from the color matrix may shed light on the meaning and function of genetic information and improve diagnostic technologies. However, the regularities have not been found yet.

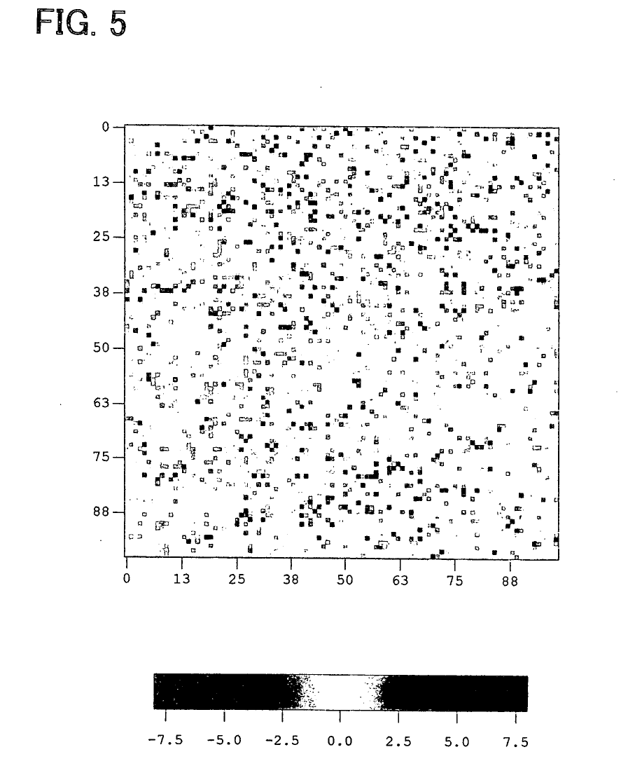

FIG. 5 shows a matrix that is the same type of matrix as FIG. 4. However, the melanoma RNA that was used in the example of FIG. 5 was extracted from a specimen that is different from the specimen that was used in the example of FIG. 4. As is clear from FIGS. 4 and 5, although the same melanoma RNA was used, the different specimen resulted in different color matrices.

Thousands of melanoma specimens or tens of thousands of melanoma specimens exist. It is difficult to extract common characteristics from the thousands or tens of thousands of matrices.

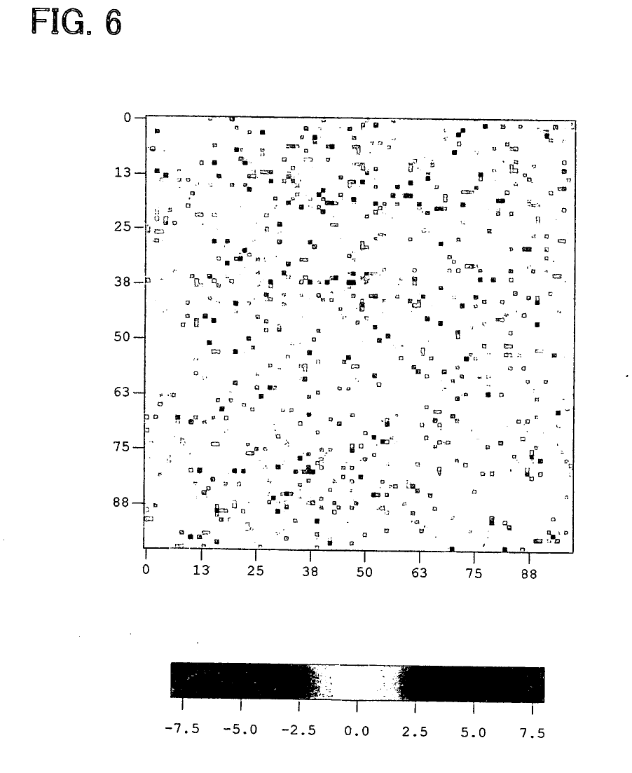

FIG. 6 shows an example of a display in which the dot color was determined for melanoma RNAs extracted from six specimens. In this case, the dot color was determined by the average of the red intensity/the green intensity ratios, which are listed in column 28 of FIG. 1. Even though the average color matrix of FIG. 6 was compared with the color matrix of FIG. 3 and with the color matrix of FIG. 4, significant characteristics could not be extracted.

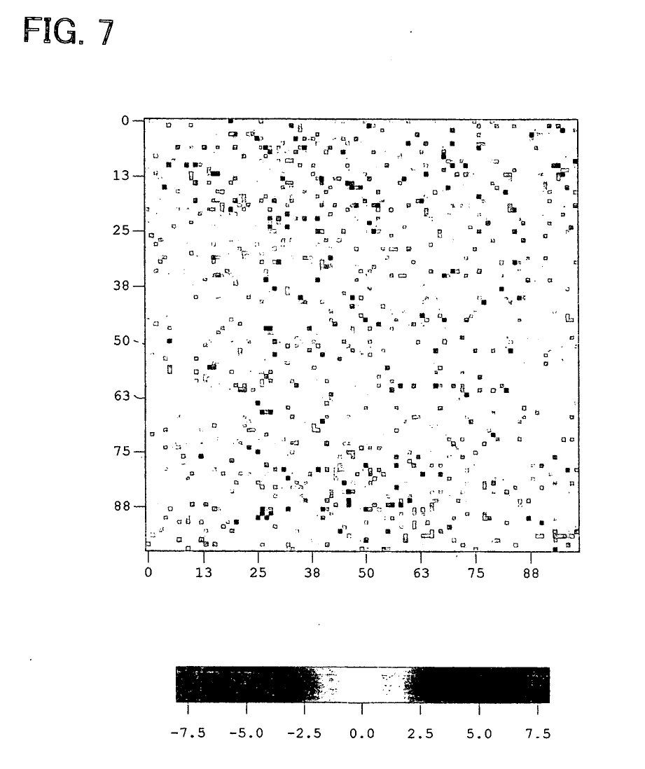

FIG. 7 shows a color matrix that was obtained from an array hybridized with RNA extracted from a colon cancer cell. Herein, conditions for the array (e.g., an array type) are the same as the case of FIG. 4.

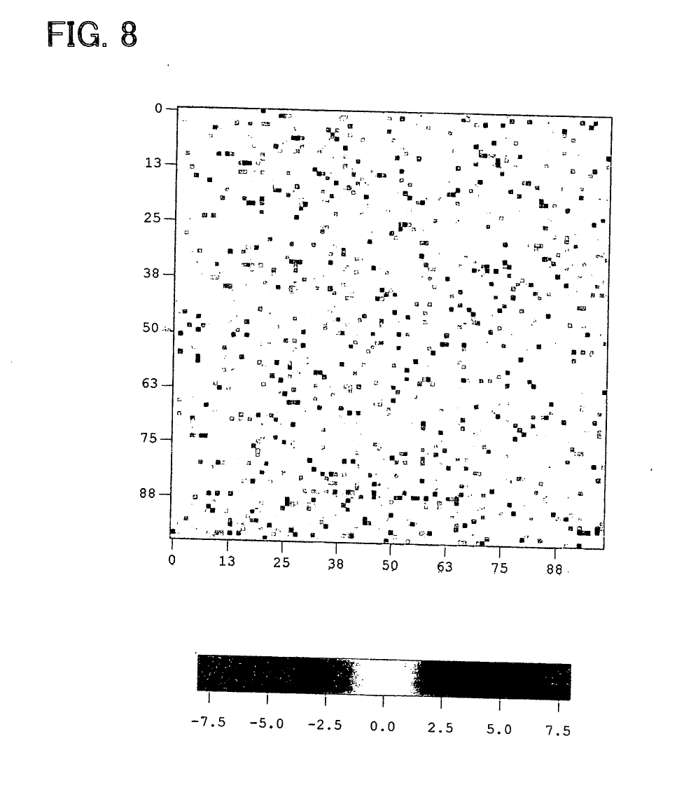

FIG. 8 shows a matrix that is the same type of matrix as FIG. 7. However, colon cancer RNA was extracted from a specimen that was different from the case of FIG. 7. Obviously, the different specimen resulted in a different color matrix, even though the same colon cancer RNA was used.

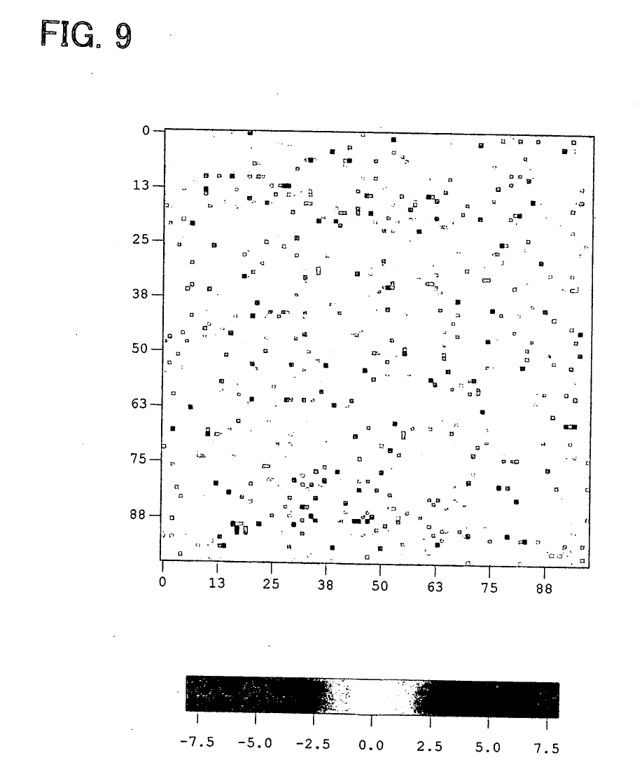

FIG. 9 shows an example of a display in which the dot color was determined for colon cancer RNAs extracted from six specimens. The dot color was determined by the average of red intensity/green intensity ratios, which are listed in column 28 of FIG. 1. Even though the average color matrix of FIG. 9 was compared with the color matrix of FIG. 7 and with the color matrix of FIG. 8, significant characteristics of the colon cancer RNAs could not be extracted.

Of the six color-matrices that are respectively shown in FIGS. 4 to 9, three of the matrices are directed to melanoma and the other three are directed to colon cancer. If characteristics that are common to the melanoma but are not common to the colon cancer or characteristics that are common to the colon cancer but are not common to the melanoma can be extracted from the six color-matrices, the extracted characteristics will directly contribute to cancer diagnoses. In addition, the extracted characteristics will enable the production of DNA microarrays that will serve as cancer markers. Further, the extracted characteristics will lead to progress in the study of cancer manifestation patterns. However, significant characteristics could not be obtained from the color matrices of FIGS. 4 to 9.

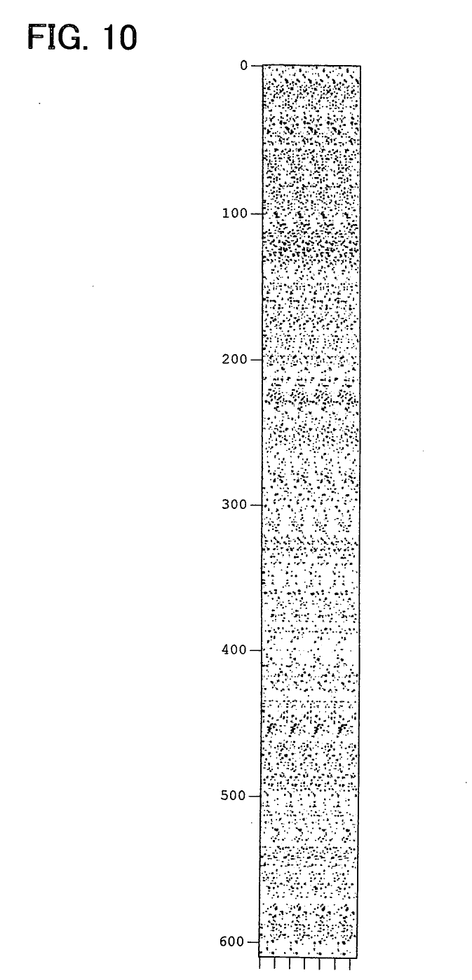

FIG. 10 shows a display obtained by converting the color matrix (98 columns·99 rows) of FIG. 4 into a color matrix (16 columns·607 rows) and then repeatedly positioning the color matrix (16 columns·607 rows) horizontally so that the matrices are parallel with each other.

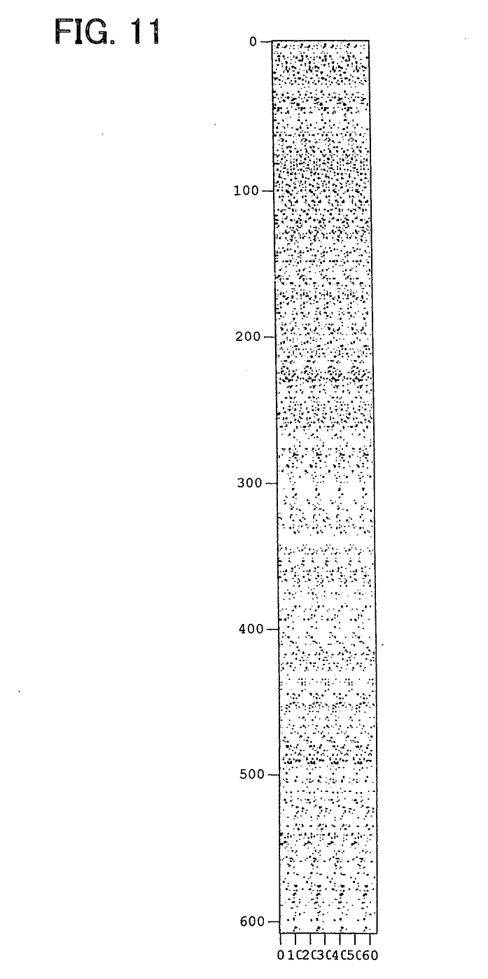

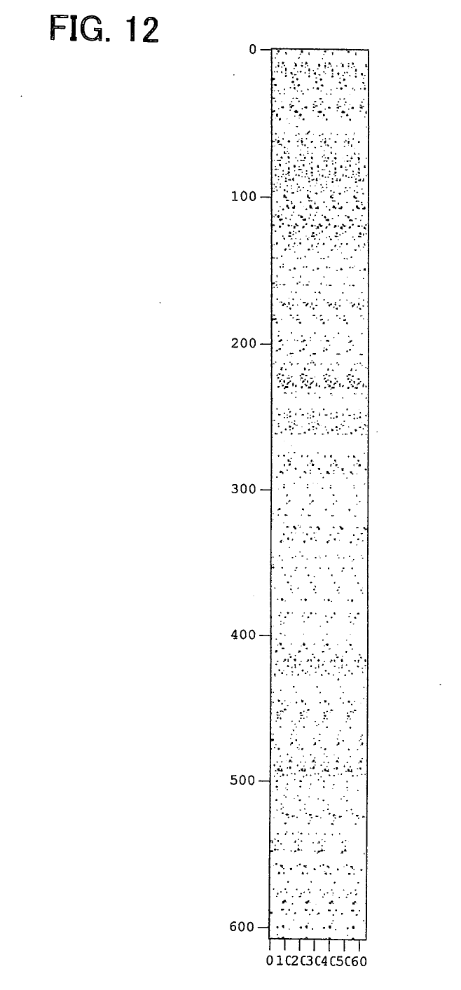

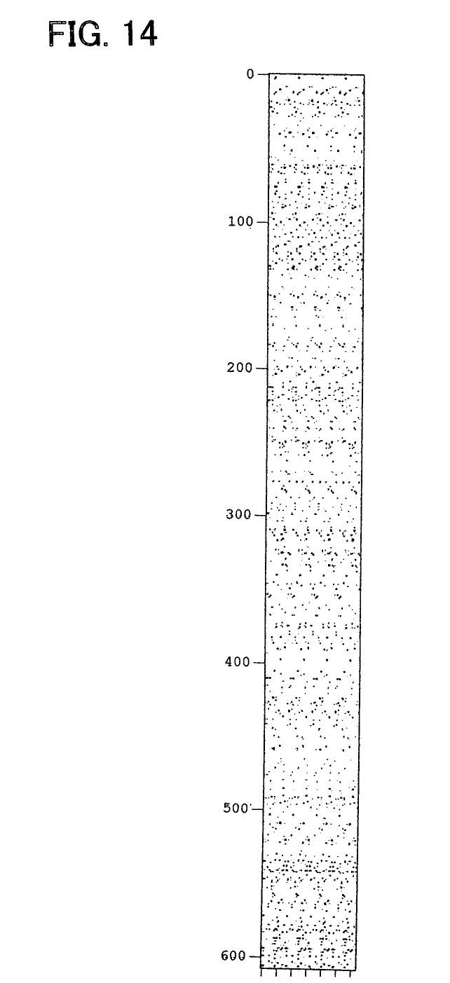

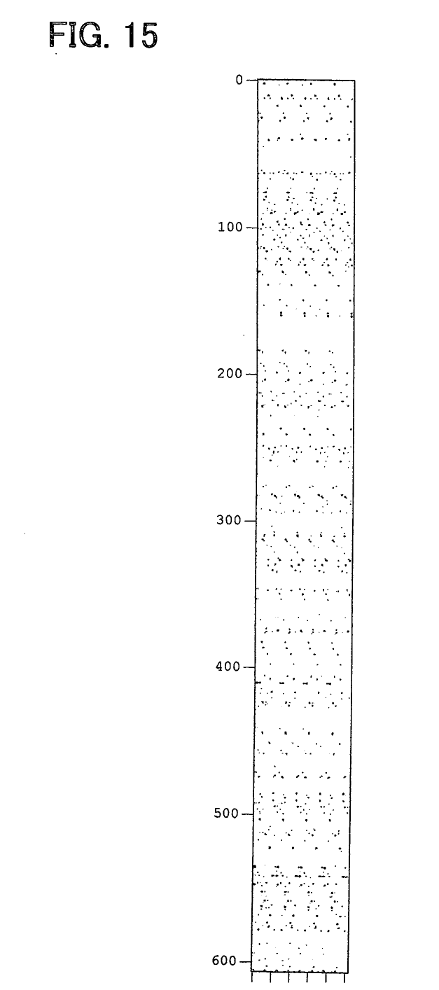

Similarly, FIGS. 11, 12, 13, 14, and 15 respectively correspond to FIGS. 5, 6, 7, 8, and 9.

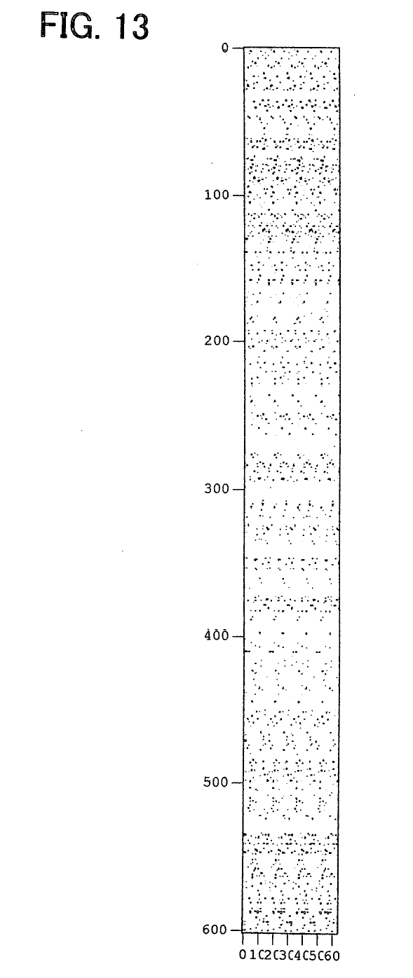

By carefully comparing the six matrices of FIGS. 10 to 15 with each other, a characteristic of each matrix can be recognized to a certain extent. For instance, the six matrices may be divided into a group of matrices of FIGS. 10 to 12 and a group of matrices of FIGS. 13 to 15. However, a difference cannot be distinctly determined between the two groups. Moreover, because each matrix is formed by simply repeating the same pattern, it is difficult to determine in which part of the matrix a characteristic exists.

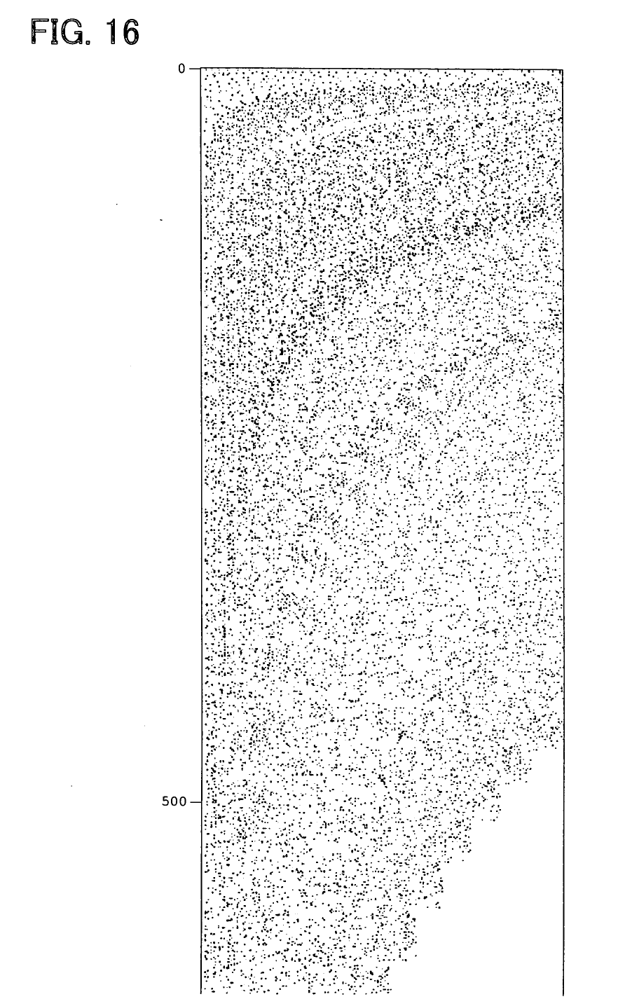

FIG. 16 shows a display obtained by converting the color matrix (98 columns·99 rows) of FIG. 4 into a display in which color matrices (3 columns·3234 rows), (4 columns·2426 rows), (5 columns·1941 rows), • • • are positioned in parallel to each other.

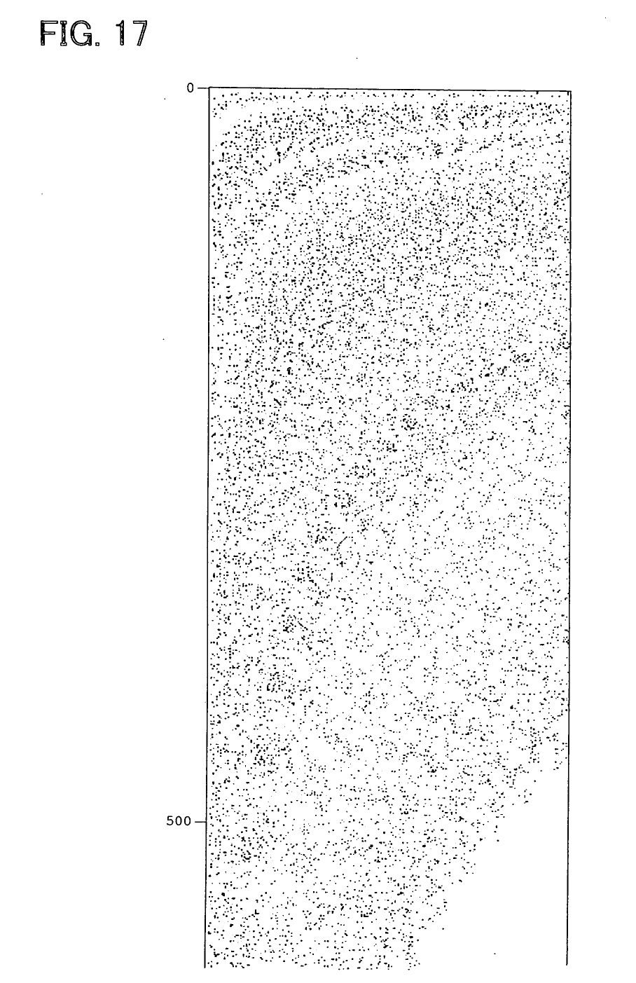

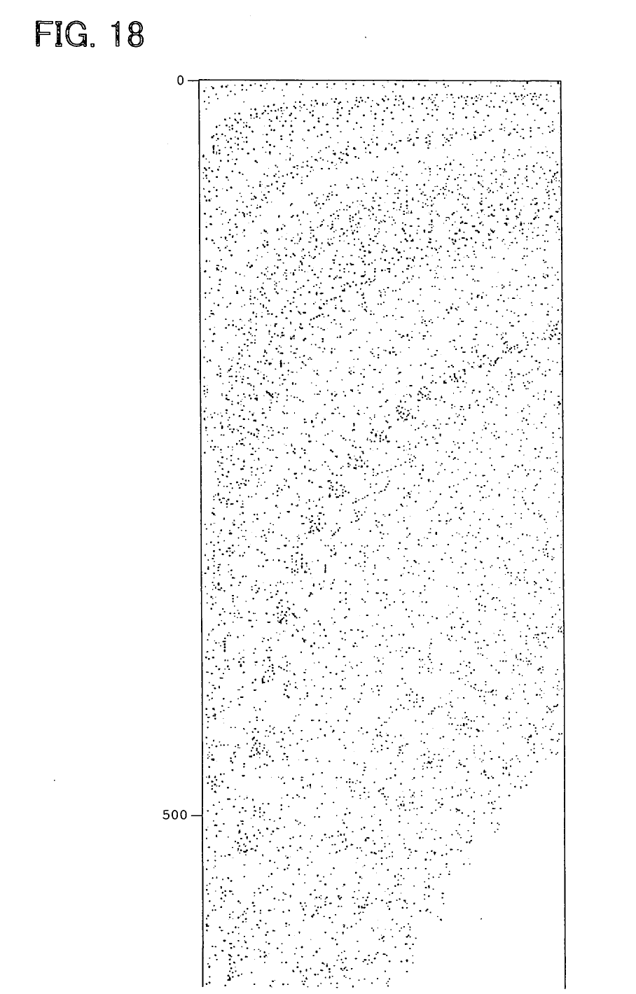

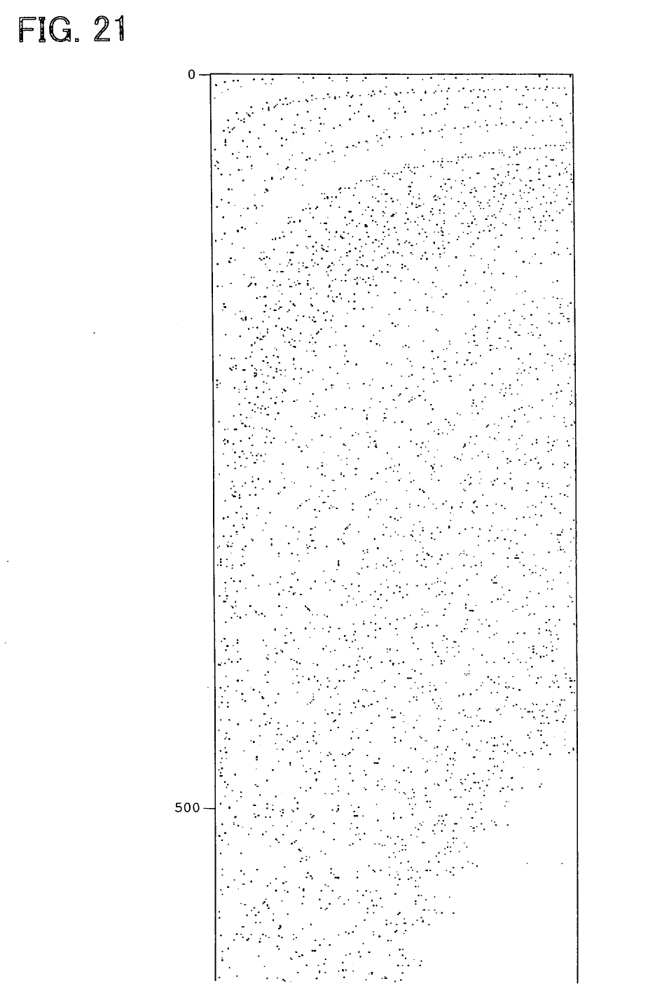

Similarly, FIGS. 17, 18, 19, 20, and 21 respectively correspond to FIGS. 5, 6, 7, 8, and 9.

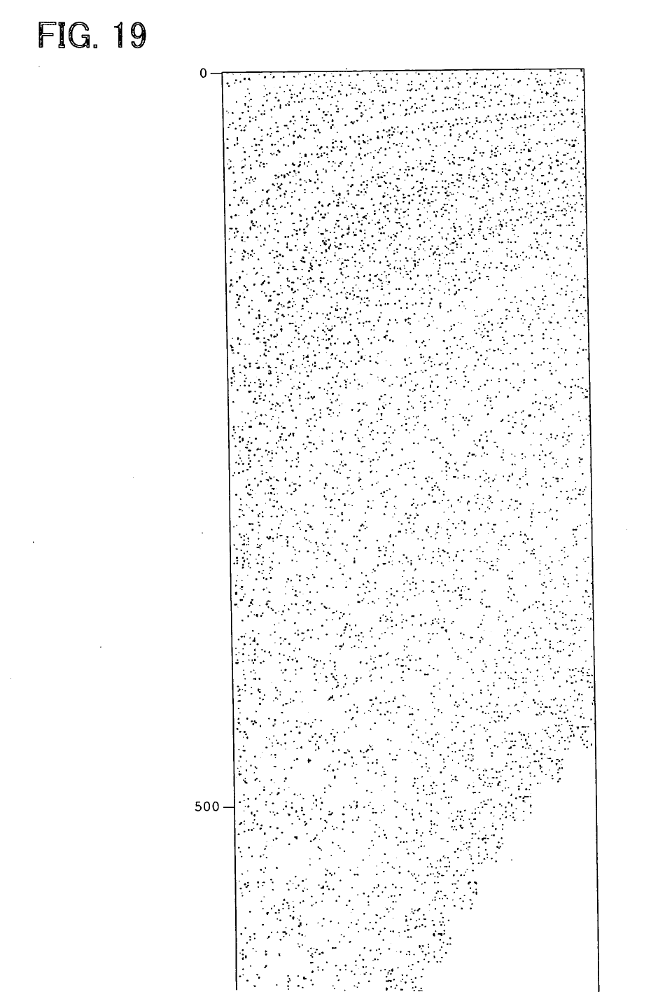

By carefully comparing the six matrices of FIGS. 16 to 21 with each other, recognition of the characteristics of the matrices becomes easier. The six matrices can be distinctively divided into a group of FIGS. 16 to 18 and a group of FIGS. 19 to 21.

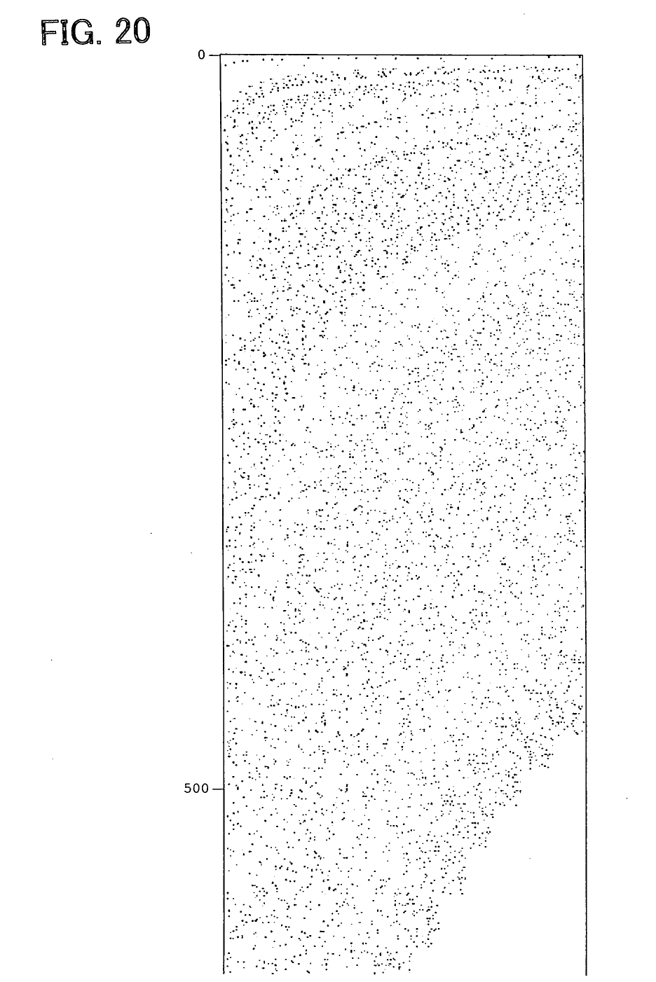

Each of the matrices of FIGS. 16 to 18, which correspond to melanoma, exhibits a distinct red streak in about the middle portion of the matrix. Each of the matrices of FIGS. 19 to 20, which correspond to colon cancer, exhibits a distinct red streak at the bottom of the matrix.

The inventor tested a certain number of people by using the set of six color matrices of FIGS. 4 to 9 (three from melanoma and the other three from colon cancer), the set of six color matrices of FIGS. 10 to 15 (three from melanoma and the other three from colon cancer), and the set of six color matrices of FIGS. 16 to 21 (three from melanoma and the other three from colon cancer). The test was conducted in order to determine how many people could correctly divide each set of matrices into two groups. The test results were as follows: a few of the people were able to divide the set of matrices of FIGS. 4 to 9 into two; nearly half of the people were able to divide the set of matrices of FIGS. 10 to 15 into two; and almost all of the people were able to divide the set of matrices of FIGS. 16 to 21 into two.

During the investigation to determine whether or not targets have hybridized with probes arranged on an array, the conversion of the original color-matrix to a parallel arrangement of color matrices that have different numbers of columns, as shown in FIG. 16, is apparently very useful. In addition, such a conversion will accelerate the discovery of manifestation patterns that appear in certain types of non-normal genes but do not appear in normal genes. Accordingly, the technology for determining whether genes are non-normal or not and the study of causes of the non-normal genes will also be advanced.

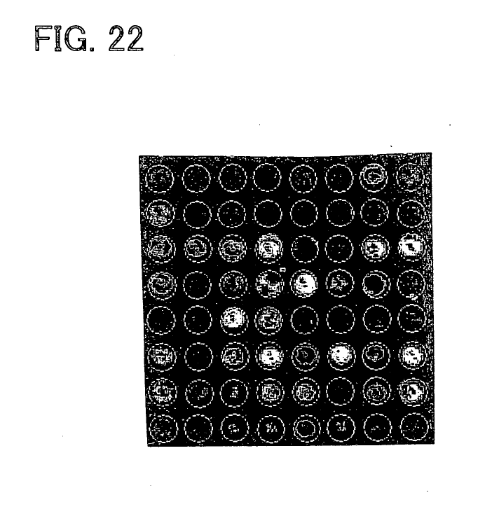

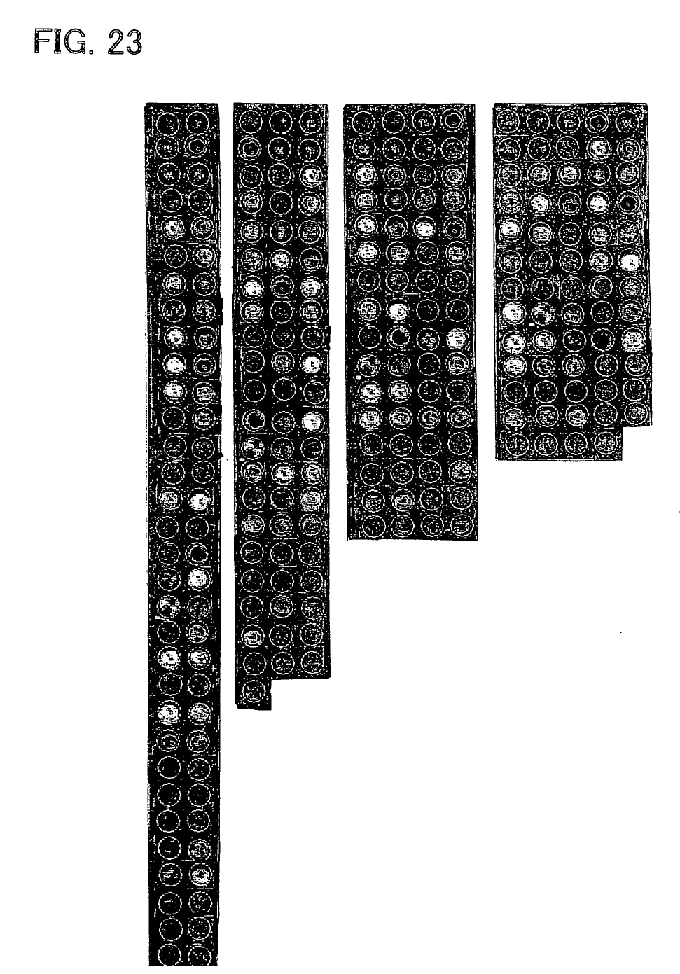

In FIGS. 4 to 21, the average color of each spot is utilized. However, the color distribution of each spot may be utilized when the color matrices that have different numbers of columns are arranged in parallel to each other. FIG. 22 shows an arrangement of spots on an array and the color distributions of the individual spots. FIG. 23 shows an example of a display in which the matrix of FIG. 22 is converted into a parallel arrangement of color matrices (2 columns·32 rows), (3 columns·22 rows), (4 columns·16 rows), and (5 columns·13 rows).

1. A method for facilitating analysis of an array after hybridization of targets with the array, in which probes are two-dimensionally disposed in the form of spots on a substrate, the method comprising:
displaying information concerning colors of the spots on the hybridized array such that at least one color matrix formed from n columns (n is a natural number), one color matrix formed from n+k columns (k is a natural number), and one color matrix formed from n+2k columns are arranged in parallel to each other.

2. A method according to claim 1, characterized in that the color information of the spots is stored in a storage means of a computer, and a matrix of color dots is generated by the computer based upon the stored data.

3. A method according to claim 2, characterized in that the matrix of color dots is printed by a printer.

4. A method according to claim 1, characterized in that the matrix of color dots is displayed by using color dots corresponding to the spots.

5. A method according to claim 1, characterized in that the matrix of color dots is displayed by using color dots corresponding to pixels of the spots.

6. A method according to claim 1, characterized in that a unit dot of the color matrix is displayed in the shape of a quadrangle.

7. A method according to claim 1, characterized in that all the dots of the color matrices that are arranged in parallel to each other have the same size.

8. A method according to claim 1, characterized in that each of the colors is selected from a spectrum ranging from red to blue based upon a (red intensity)/(red intensity+green intensity) ratio of the corresponding spot that was hybridized with the targets of two types, the target of one of the types being colored in red and the target of the other type being colored in green, and the selected colors corresponding to the spots are displayed in the shape of quadrangles and in rows and columns of the matrix.

9. An apparatus for facilitating analysis of a hybridized array, the apparatus comprising:
means for storing color information of spots on the hybridized array, and
means for generating a display from the stored color information, in which at least one color matrix formed from n columns (n is a natural number), one color matrix formed from n+k columns (k is a natural number), and one color matrix formed from n+2k columns are arranged in parallel to each other.
